(19) United States
(12) Patent Application Publication
(10) Pub. No.: US 2025/0025140 A1
(43) Pub. Date: Jan. 23, 2025

(54) FLEXIBLE CORE NEEDLE FOR BRONCHOSCOPY

(71) Applicant: Michael J. Simoff, Northville, MI (US)

(72) Inventor: Michael J. Simoff, Northville, MI (US)

(21) Appl. No.: 18/713,628

(22) PCT Filed: Nov. 15, 2022
(86) PCT No.: PCT/US2022/049941

§ 371 Date: May 24, 2024

Related U.S. Application Data
(60) Provisional application No. 63/286,253, filed on Dec. 6, 2021.

(51) Int. Cl. A61B 10/02; A61B 10/04

(52) U.S. Cl. CPC A61B 10/0275; A61B 10/0283; A61B 10/04; A61B 2010/0208; A61B 2010/045

(57) ABSTRACT

A flexible core needle, which may be used with an endoscope, and has an insertion tube defining a working channel. The flexible core needle includes a housing defining a sample chamber and an internal blade selectively movable between a closed position and an open position to collect a lesion sample. The flexible core needle may have an actuator passing within a flexible feeder, such that the actuator controls movement of the internal blade between the closed position and the open position. Suction may be applied while the internal blade is open to collect the lesion sample. Methods of using the flexible core needle to collect samples are also presented.

### CROSS-REFERENCE TO RELATED APPLICATIONS

This application claims the benefit of U.S. Provisional Application No. 63/286,253, filed Dec. 6, 2021, which is hereby incorporated by reference in its entirety.

### INTRODUCTION

This disclosure generally relates to flexible core needles usable for collecting tissue samples, such as biopsies.

### SUMMARY

A flexible core needle, which may be used with an endoscope, and has an insertion tube defining a working channel. The flexible core needle includes a housing defining a sample chamber and an internal blade selectively movable between a closed position and an open position, within the sample chamber, to collect a lesion or tissue sample.

The flexible core needle may have an actuator passing within a flexible feeder, such that the actuator controls movement of the internal blade between the closed position and the open position. The flexible feeder may be operatively attached to the housing and is passable through the working channel of the endoscope and is selectively extendable therefrom.

The flexible core needle may have a suction device configured to selectively apply a vacuum within the sample chamber while the internal blade is substantially in the open position, such that the suction device communicates with the sample chamber through a portion of the flexible feeder. A handle may cooperate with the flexible core needle and includes, at least, a base arm, a pivot mechanism operatively attached to the base arm, and a moveable arm operatively attached to the base arm via the pivot mechanism. The flexible feeder is operatively attached to the base arm, and the actuator is operatively attached to the moveable arm, such that the internal blade is moved between the closed position and the open position by the moveable arm. The suction device is operatively attached to the base arm, such that the vacuum is applied to the flexible feeder via the base arm.

A method of collecting tissue samples with a flexible core needle is also provided. The method includes: moving a tip of an endoscope to a location proximate a lesion; advancing the flexible core needle, with an internal blade closed within a housing; opening the internal blade to expose a sample chamber of the housing; drawing a histologic sample of the lesion into the sample chamber with the applied suction; closing the internal blade with the histologic sample contained therein; and retracting the flexible core needle, with the collected histologic sample within the sample chamber and the internal blade. The method may apply suction to the housing before closing the internal blade. Note that the order of the method steps is not limiting.

The above features and advantages, and other features and advantages, of the present disclosure are readily apparent from the following detailed description of some of the best modes and other embodiments for carrying out the disclosure, which is defined solely by the appended claims, when taken in connection with the accompanying drawings.

### BRIEF DESCRIPTION OF THE DRAWINGS

FIG. 1 schematically illustrates an isometric view of a flexible core needle shown in a closed position.

FIG. 2 schematically illustrates an isometric view of the flexible core needle shown in a partially open position.

FIG. 3 schematically illustrates another isometric view of the flexible core needle shown in a fully open position and illustrated relative to an endoscope.

FIG. 4 schematically illustrates another isometric view of the flexible core needle shown in a partially open position.

FIG. 5 schematically illustrates a side view of the flexible core needle shown in a fully open position.

FIG. 6 schematically illustrates a side view of the flexible core needle shown in a partially open position.

FIG. 7 schematically illustrates a side view of the flexible core needle shown in a partially open position, with internal components partially illustrated in dashed lines, including an interior blade, an O-ring, and an actuator.

FIG. 8 schematically illustrates a top view of the flexible core needle shown without the interior blade and actuator.

FIG. 9 schematically illustrates a top view of the flexible core needle shown without the interior blade and actuator, with internal components partially illustrated in dashed lines.

FIG. 10 schematically illustrates a handle for the flexible core needle, including a suction device.

FIG. 11 schematically illustrates the handle for the flexible core needle, with internal components partially illustrated in dashed lines.

### DETAILED DESCRIPTION

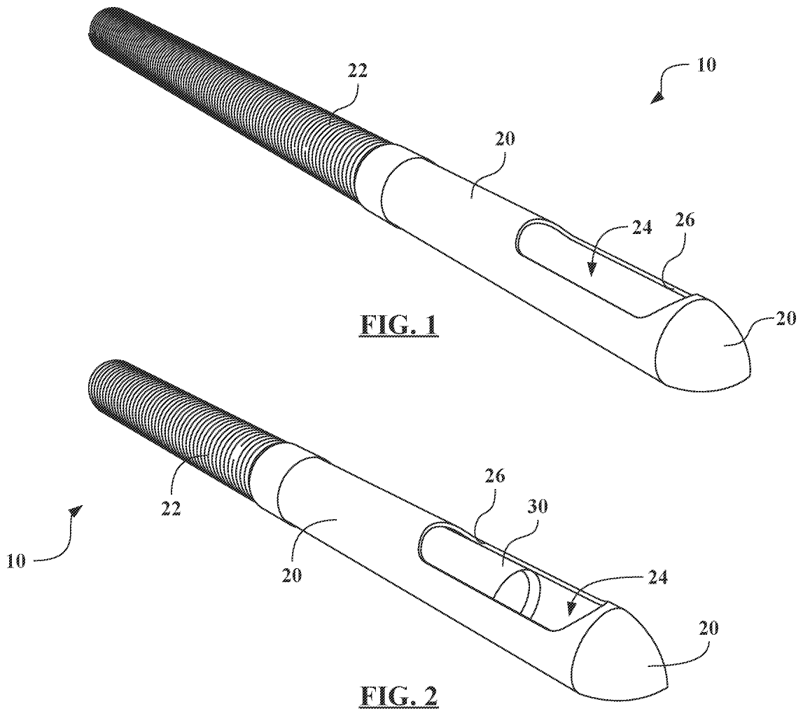

Referring to the drawings, like reference numbers correspond to like or similar components wherever possible throughout the several figures. All figures may be referred to in any section of the specification, without regard to numerical order. As shown in FIGS. 1-4 there are various isometric views of a flexible core needle 10 that may be used with an endoscope 12.

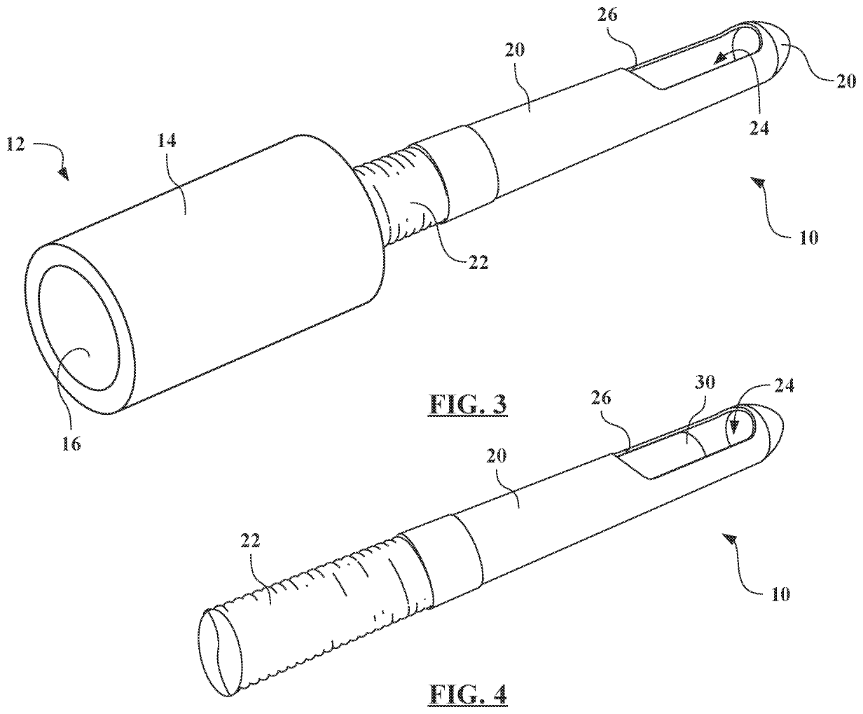

The flexible core needle 10, which may simply be referred to as needle 10, may be used as biopsy device or biopsy tool. FIG. 1 shows the flexible core needle 10 in a fully closed position, FIG. 3 shows the flexible core needle 10 in a fully open position, and FIGS. 2 and 4 show the flexible core needle 10 in partially open positions.

The portion of the endoscope 12 viewable in the figures is an insertion tube 14, which is shown highly schematically and extends much further leftward (as viewed in FIG. 3) relative to the flexible core needle 10. The endoscope 12 is flexible and the insertion tube 14 of the endoscope 12 defines an instrument channel or working channel 16, through which the flexible core needle 10 may be inserted. The endoscope 12 is of sufficient design and length to pass through the nose or mouth and is capable of accessing the airways of the lungs. The working channel 16 passes through the length of the endoscope 12. It allows the passage of tools safely through the length of the endoscope 12 when in place within the lungs.

Those having ordinary skill in the art will recognize numerous types and configurations for the endoscope 12, which may be referred to as a bronchoscope, usable with the flexible core needle 10 described herein. The working channel 16 ends at a tip, beyond which the flexible core needle 10 is selectively extendable. Note that the flexible core needle 10 would extend leftward (as viewed in the figures) through the working channel 16 of the insertion tube 14, but that portion is not illustrated to better illustrate the working channel 16 through which the flexible core needle 10 passes.

The flexible core needle 10 includes a housing 20 attached to a flexible feeder 22. The housing 20 may also be referred to as the piercing housing 20, as it includes a point configured to, when necessary, pierce portions of the biopsy site. The housing 20 is substantially hollow and defines a sample port or sample chamber 24 therein. The sample chamber 24 is accessible through an opening 26 in the housing 20.

The flexible feeder 22 is also substantially hollow. The flexible feeder 22 and the housing 20 are inserted through the working channel 16 of the endoscope 12, which is controlled to be near the site for the biopsy and provides a substantially rigid or solid platform for the flexible core needle 10. The robotic endoscope 12 may allow the tip of the scope to be within 5-25 mm of the tumor, such that the flexible core needle 10 is proximate the lesion needing to be sampled.

The flexible core needle 10 includes an internal blade 30, which is selectively movable between opened and closed positions, and partial positions, within the sample chamber 24 of the housing 20. FIG. 1 shows the internal blade 30, which may be referred to simply as the inner blade 30 or the blade 30, fully closed within the flexible core needle 10; FIG. 2 and FIG. 4 show the internal blade 30 partially open or retracted; and FIG. 3 shows the internal blade 30 in a fully open or retracted position. Note that the internal blade 30 includes an interior cylindrical opening

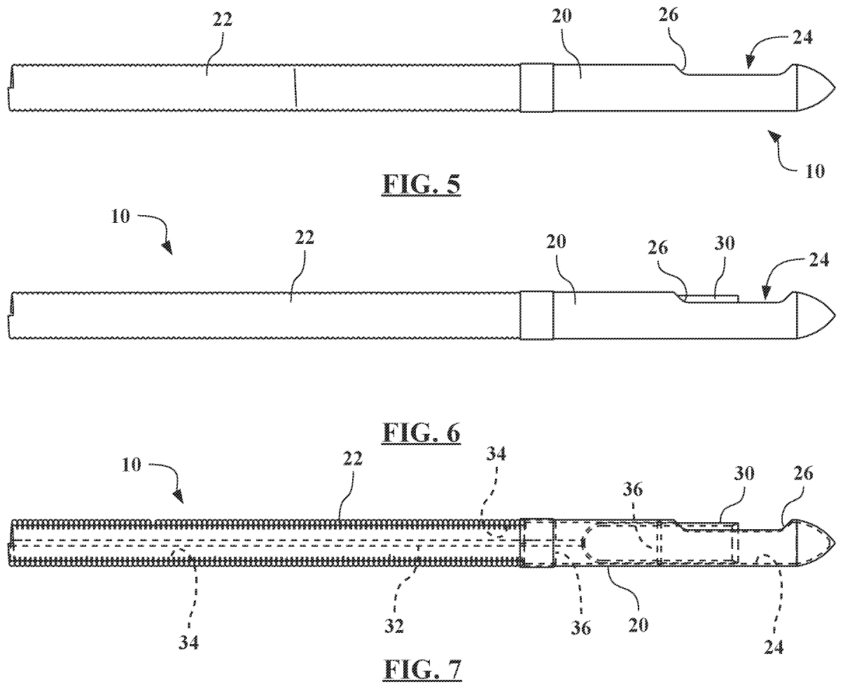

FIGS. 5-7 show side views of the flexible core needle 10 and illustrates the internal blade 30. FIG. 5 shows the internal blade 30 fully retracted, FIG. 6 shows the internal blade 30 partially retracted, and FIG. 7 shows the internal blade 30 partially retracted with the interior components in dashed lines. As best viewed in FIG. 7, an actuator 32 is operatively attached to the internal blade 30 and is configured to selectively move the internal blade 30. Surrounding the actuator 32 within the flexible feeder 22 is a vacuum chamber 34, through which an operator may selectively apply a vacuum or suction. One or more O-rings 36 hold, support, and/or guide the internal blade 30 within the housing 20.

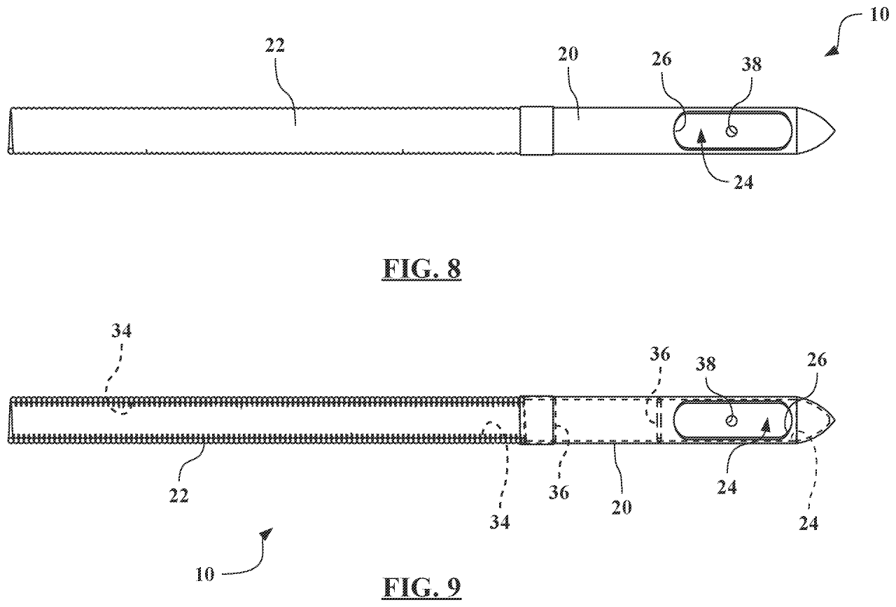

FIGS. 8-9 show top views of the flexible core needle 10 with the internal blade 30 removed. FIG. 9 shows some of the interior components in dashed lines. As shown in FIGS. 8 and 9, the flexible core needle 10 may include a knock-out hole 38, or simply hole 38, formed in the housing 20. The hole 38 may be used to remove the collected architectural histologic sample by inserting a small device through the hole to push the sample out of the sample chamber 24. Some configurations of the flexible core needle 10 may not include the hole 38.

Histology refers to tissue structure or organization, including its architecture. The flexible core needle 10 is capable of collecting a histologic sample that retains the sampled tissue's architecture. Whereas cytology refers to examination of cells obtained, such as by aspiration or scraping, for diagnostic purposes, without architectural integrity. Alternative needles are cytologic needles that are only capable of collecting cytologic samples.

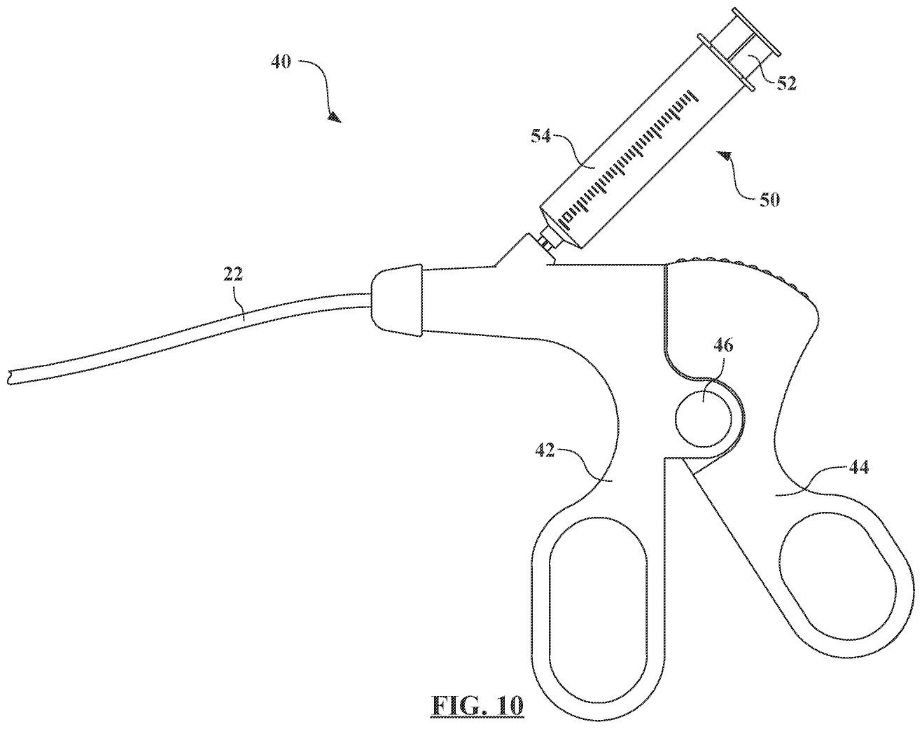

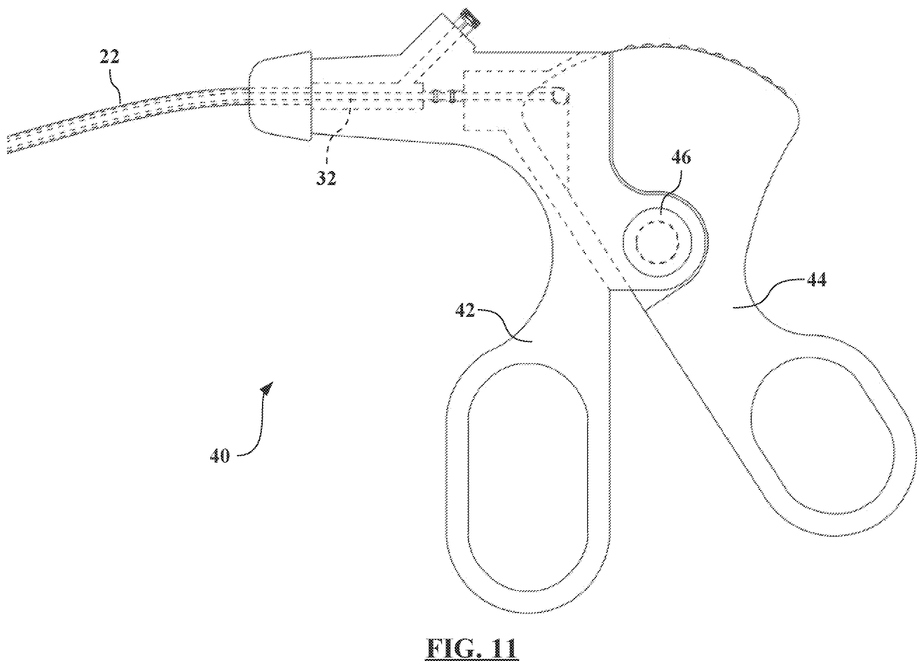

Referring also to FIGS. 10 and 11, the biopsy device or flexible core needle 10 may be triggered or controlled by a handle 40, which is also novel. The handle 40 is shown schematically in FIG. 10 and is also shown in FIG. 11 with some interior portions or features shown in dashed lines. Skilled artisans will recognize use of the handle 40 with the endoscope 12 to selectively operate the flexible core needle 10 described herein. The flexible endoscope 12 may be robotic, manually operated, or otherwise controlled.

The handle 40 includes a fixed portion, fixed or base arm, or base 42 and a moveable arm 44 attached to the base 42 via a pivot mechanism 46. The flexible feeder 22 is operatively attached to the handle 40, and a portion of the actuator 32 is attached to the moveable arm 44 opposite the pivot mechanism 46.

As best viewed in FIG. 11, rotation of the moveable arm 44 clockwise (as viewed in the figures) about the pivot mechanism 46 will cause the internal blade 30, which is attached to the actuator 32, to retract within the sample chamber 24 and open the flexible core needle 10. Similarly, counterclockwise rotation of the moveable arm 44, which may occur as a result of a spring mechanism or movement by the operator, will cause the internal blade 30 to advance back into the sample chamber 24, closing the flexible core needle 10.

A suction device 50 is attached to the handle 40 and is operatively connected to the vacuum chamber 34 of the flexible feeder 22, which is operatively connected to the sample chamber 24. In the configuration shown, the suction device 50 connects to the vacuum chamber 34 via an internal chamber or connection within the base 42. For example, and without limitation, the vacuum chamber 34 may be connected to the sample chamber 24 through a hole in the internal blade 30 or a channel/passage along the inside of the housing 20. To create suction within the vacuum chamber 34, a plunger 52 is partially retracted from a cylinder 54. In some configurations, the suction device 50 may be lockable while applying suction, such as, for example, by turning suction device 50.

Alternative needles used for collection of samples may utilize transthoracic fine needle aspiration (FNA), which usually collects a cytologic sample. The transthoracic needle must be inserted through the skin, across both layers of the pleural, and into the lung tissue, and has a high risk for pneumothorax (collapsed lung). The flexible core needle 10 is capable of collecting a histologic sample that retains the sample architecture. The flexible core needle 10 techniques provide a sample with defined architecture, which is needed for modern molecular assessment of samples, the diagnosis of lymphomas, and, also, may be required for patients to be included into clinical trials.

Most alternative needles collect cytologic—i.e., single cell type—samples, which do not retain the architecture of the sample. With cytologic samples, a diagnosis is possible, but the cytologic sample lacks key characteristics of the tissue needed to offer certain treatments. This generally increases the need to collect multiple samples, which is inefficient for analysis and diagnosis, and, also, economically inefficient.

For the alternative needle collection, the transthoracic needle is inserted through the back or side, and the needles must be pushed into the lesion. This process generally requires the use of CT scanning with increased radiation. Then, with suction applied, moved in an in-and-out fashion for a number of passes. This makes the sample retrieved liquified, such that it is difficult to determine the type of tissue collected and there is no architectural integrity. A diagnosis may be made cytologically with this type of sample (if adequate) but is less beneficial. The likely result is a sample that is identified as probably malignant but needs further sampling to determine the location/type of malignancy.

The histologic sample collected by the flexible core needle 10 overcomes these deficiencies and provides a sample that retains architectural integrity, which is better for analysis and proper, or full, diagnosis. Additional information, such as molecular markers, immunologic markers, and histology requires architectural integrity. The new tool of the flexible core needle 10 allows single procedures to provide all information necessary.

The alternative back-and-forth motion of the transthoracic needle is blind and arbitrary, without real-time visualization, based on information gleaned from prior guidance information and/or CT scanning. Due to the fact that sampling could potentially take place completely outside of the lesion, due to the back-and-forth or in-and-out motion necessary for sampling, this can interfere with the accuracy of performing the resulting biopsy.

For example, and without limitation, there are generally four ways of accessing a nodule for biopsy via endoscopy, such as may be used with the flexible core needle 10. The first is a robotic system and the second is a manual system, where, using a bronchoscope, the device is inserted through and into the periphery of the lung, typically using fluoroscopy. The third is called electromagnetic navigation (EMN), and the fourth is called radial endobronchial ultrasound (rEBUS) guided procedure.

With EMN, a navigational catheter is inserted through a bronchoscope or endoscope, with which the operator can partially control the direction of the tip towards the lesion. Once guided to the location of the lesion, the navigational catheter is removed and an extendable biopsy channel (a hollow catheter) is left in place, through which the flexible core needle 10 and/or other tools are placed. With rEBUS, a radial ultrasound probe with a catheter or sheath is passed through a manual bronchoscope and pushed out into the lung. When the lesion is identified, the rEBUS probe is pulled out through the guide sheath, which is left in place, and a needle, such as the flexible core needle 10, can be passed through. Manual systems of bronchoscopy will be recognized by those having ordinary skill in the art.

The flexible core needle 10, as delivered and aligned by the endoscope 12, may be used to collect tissue samples, such as those used to test for one or more diseases. For example, and without limitation, the flexible core needle 10 may be used to perform a biopsy of a lesion or tumor to evaluate for lung cancer.

Bronchoscopy—the use of a fiberoptic scope inserted through the mouth or nose of a patient—is one technique to access lung nodules for the diagnosis of lung cancer. The traditional procedure of bronchoscopy used to sample a peripheral lesion is a transbronchial biopsy, which uses forceps directed out into the lung to biopsy a lesion. The endoscope 12 may be a robotic bronchoscope, which provides a stable and sensitive platform for use of the flexible core needle 10. The robotic endoscope 12 can be used to guide the flexible core needle 10 to, and localize, peripheral tumors to a higher percentage of capture.

A variety of imaging technologies are used in the assessment of patients with suspected lung cancer often identifying sites of disease or assisting in choosing targets for biopsy. These technologies may be used in conjunction with the endoscope 12 and the flexible core needle 10 described herein.

While the present disclosure may be illustrated with respect to particular industries or applications, those skilled in the art will recognize the broader applicability of the products, methods, and techniques, described herein. For example, similar structures, methods, or combinations thereof, may be used in other industries or for medical procedures other than those described herein. The order of any method steps described herein is not limiting.

Those having ordinary skill in the art will also recognize that terms such as “above,” “below,” “upward,” “downward,” et cetera, are used descriptively of the figures, and do not represent limitations on the scope of the appended claims. Any numerical designations, such as “first” or “second” are illustrative only and are not intended to limit the scope of the claims in any way.

When used herein, the term “substantially” refers to relationships that are ideally perfect or complete, but where manufacturing realties prevent absolute perfection. Therefore, substantially denotes typical variance from perfection in the relevant art. For example, if height A is substantially equal to height B, it may be preferred that the two heights are 100.0% equivalent, but manufacturing realities likely result in the distances varying from such perfection. Skilled artisans would recognize the amount of acceptable variance. For example, and without limitation, coverages, areas, or distances may generally be within 10% of perfection for substantial equivalence. Similarly, relative alignments, such as parallel or perpendicular, may generally be within 5%.

Features shown in one figure may be combined with, substituted for, or modified by, features shown in any of the figures. Unless stated otherwise, no features, elements, or limitations are mutually exclusive of any other features, elements, or limitations. Any specific configurations shown in the figures are illustrative only and the specific configurations shown are not limiting.

The novel flexible core needle 10 presented allows for the ability to pass into a lesion and, with the addition of suction and the closing of the internal blade 30, acquire a core sample for diagnosis without the in-and-out motion required by alternative needles. One example, without limitation, of operation of flexible core needle 10 to collect a sample is described herein.

The endoscope 12 guides, possibly via robotics, the flexible core needle 10 nearby the lesion and provides a stable base or platform for the flexible core needle 10 to biopsy the lesion. The flexible core needle 10 is inserted into the lesion with the internal blade 30 closed. Suction is applied by the suction device 50 as the internal blade 30 is retracted, or after the internal blade 30 is retracted, by movement of the moveable arm 44 of the handle 40.

The applied suction pulls a full histologic sample, which maintains intact tissue architecture, into the sample chamber 24 of the flexible core needle 10. The internal blade 30 may then be closed by the moveable arm 44, capturing the histologic sample within the sample chamber 24, and the flexible core needle 10 retracted through the endoscope 12.

Following retraction of the flexible core needle 10 through the endoscope 12, the histologic sample, with architecture intact, may be removed from the sample chamber 24. Removal may include using the hole 38, or other mechanisms to maintain the architecture collected by the flexible core needle 10.

In some configurations, the flexible core needle 10 may be used with devices other than the endoscope 12 or a robotic bronchoscope and/or may be used for sampling lesions not involved with the lungs. For example, and without limitation, the flexible core needle 10 may be used for gastrointestinal procedures (GI) to sample lesions within the GI tract.

### Clauses:

A flexible core needle usable with an endoscope having an insertion tube defining a working channel, the flexible core needle comprising: a housing defining a sample chamber; and an internal blade selectively movable between a closed position and an open position, within the sample chamber, to collect a lesion sample.

The flexible core needle of any clause, further comprising: a flexible feeder operatively attached to the housing, wherein the flexible feeder is passable through the working channel of the endoscope and is selectively extendable therefrom.

The flexible core needle of any clause, further comprising: an actuator passing within the flexible feeder, wherein the actuator controls movement of the internal blade between the closed position and the open position.

The flexible core needle of any clause, further comprising: a suction device configured to selectively apply a vacuum within the sample chamber while the internal blade is at least partially in the open position, and wherein the suction device communicates with the sample chamber through a portion of the flexible feeder.

The flexible core needle of any clause, further comprising: a handle having: a base arm; a pivot mechanism operatively attached to the base arm; and a moveable arm operatively attached to the base arm via the pivot mechanism; wherein the flexible feeder is operatively attached to the base arm; wherein the actuator is operatively attached to the moveable arm, such that the internal blade is moved between the closed position and the open position by the moveable arm; and wherein the suction device is operatively attached to the base arm, such that the vacuum is applied to the flexible feeder via the base arm.

A method of collecting tissue samples with an endoscope and a flexible core needle, the method comprising: moving a tip of the endoscope to a location proximate a lesion; advancing the flexible core needle, with an internal blade closed within a housing, through the endoscope; opening the internal blade to expose a sample chamber of the housing; drawing a histologic sample of the lesion into the sample chamber; closing the internal blade with the histologic sample contained therein; and retracting the flexible core needle, with the histologic sample within the sample chamber and the internal blade.

The method of collecting tissue samples of any clause, further comprising: applying suction to the housing to draw the histologic sample of the lesion into the sample chamber, before closing the internal blade.

The method of collecting tissue samples of any clause, wherein the method does not involve moving the flexible core needle back and forth about the lesion.

The detailed description and the drawings or figures are supportive and descriptive of the disclosure. While some of the best modes and other embodiments for carrying out the disclosure have been described in detail, various alternative designs, configurations, and embodiments exist for practicing the appended claims, as will be recognized by those having ordinary skill in the art.

1. A flexible core needle usable with an endoscope having an insertion tube defining a working channel, the flexible core needle comprising:
a housing defining a sample chamber; and
an internal blade selectively movable between a closed position and an open position, within the sample chamber, to collect a lesion sample.

2. The flexible core needle of claim 1, further comprising:
a flexible feeder operatively attached to the housing, wherein the flexible feeder is passable through the working channel of the endoscope and is selectively extendable therefrom.

3. The flexible core needle of claim 2, further comprising:
an actuator passing within the flexible feeder, wherein the actuator controls movement of the internal blade between the closed position and the open position.

4. The flexible core needle of claim 3, further comprising:
a suction device configured to selectively apply a vacuum within the sample chamber while the internal blade is at least partially in the open position, and wherein the suction device communicates with the sample chamber through a portion of the flexible feeder.

5. The flexible core needle of claim 4, further comprising:
a handle having:
a base arm;
a pivot mechanism operatively attached to the base arm; and
a moveable arm operatively attached to the base arm via the pivot mechanism;
wherein the flexible feeder is operatively attached to the base arm;
wherein the actuator is operatively attached to the moveable arm, such that the internal blade is moved between the closed position and the open position by the moveable arm; and
wherein the suction device is operatively attached to the base arm, such that the vacuum is applied to the flexible feeder via the base arm.

6. The flexible core needle of claim 3, further comprising:
a handle having:
a base arm;
a pivot mechanism operatively attached to the base arm; and
a moveable arm operatively attached to the base arm via the pivot mechanism; and
wherein the actuator is operatively attached to the moveable arm, such that the internal blade is moved between the closed position and the open position by the moveable arm.

7. The flexible core needle of claim 1, further comprising:
a suction device configured to selectively apply a vacuum within the sample chamber while the internal blade is at least partially in the open position.

8. A flexible core needle for collecting a lesion sample comprising:
a housing defining a sample chamber;
an internal blade selectively movable between a closed position and an open position to collect the lesion sample; and
a flexible feeder operatively attached to the housing.

9. The flexible core needle of claim 8, further comprising:
an actuator passing through the flexible feeder, wherein the actuator controls movement of the internal blade between the closed position and the open position.

10. The flexible core needle of claim 9, wherein the internal blade moves within the sample chamber between the closed position and the open position.

11. The flexible core needle of claim 10, further comprising:
a handle having:
a base arm;
a pivot mechanism operatively attached to the base arm; and
a moveable arm operatively attached to the base arm via the pivot mechanism; and
wherein the actuator is operatively attached to the moveable arm, such that the internal blade is moved between the closed position and the open position by the moveable arm.

12. The flexible core needle of claim 9, further comprising:
a handle having:
a base arm;
a pivot mechanism operatively attached to the base arm; and
a moveable arm operatively attached to the base arm via the pivot mechanism; and
wherein the actuator is operatively attached to the moveable arm, such that the internal blade is moved between the closed position and the open position by the moveable arm.

13. A method of collecting tissue samples with an endoscope and a flexible core needle, the method comprising:
moving a tip of the endoscope to a location proximate a lesion;
advancing the flexible core needle, with an internal blade closed within a housing, through the endoscope;
opening the internal blade to expose a sample chamber of the housing;
drawing a histologic sample of the lesion into the sample chamber;
closing the internal blade with the histologic sample contained therein; and
retracting the flexible core needle, with the histologic sample within the sample chamber and the internal blade.

14. The method of collecting tissue samples of claim 13, further comprising:
applying suction to the housing to draw the histologic sample of the lesion into the sample chamber, before closing the internal blade.

15. The method of collecting tissue samples of claim 14, wherein the method does not involve moving the flexible core needle back and forth about the lesion.
